(19) United States
(12) Patent Application Publication
(10) Pub. No.: US 2017/0348419 A1
(43) Pub. Date: Dec. 7, 2017

(54) ORAL LIQUID SUSPENSIONS

(71) Applicant: NuBioPharma, LLC, Sanford, NC (US)

(72) Inventors: Inderjit Kumar Dev, Durham, NC (US); Ajay Kumar Ajmani, Sanford, NC (US); Ronald Lee Morris, JR., Sanford, NC (US)

(73) Assignee: NuBioPharma, LLC, Sanford, NC (US)

(21) Appl. No.: 15/174,359

(22) Filed: Jun. 6, 2016

(51) Int. Cl. A61K 47/32; A61K 31/519; A61K 9/00; A61K 9/14; A61K 47/10; A61K 31/635

(52) U.S. Cl. CPC A61K 47/32; A61K 47/10; A61K 31/635; A61K 9/0053; A61K 9/14; A61K 31/519

(57) ABSTRACT

The present invention is a novel oral formulation for the administration of either celecoxib or allopurinol which provides a useful shelf life and is easy to re-suspend.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to the use of certain novel liquid oral compositions of two medications, celecoxib and allopurinol, for the treatment of a disease in a mammal in need of celecoxib therapy or allopurinol therapy. The present invention also discloses that celelecoxib or allopurinol, glycerol, and insoluble polyvinylpyrrolidone (crospovidone) form a good aqueous suspension, having a useful shelf life and is easily re-suspended if settling occurs.

### Description of Related Art

The lack of an appropriate oral dosage formulation limits the use of many medications that may potentially benefit children. The majority of children between 2 and 11 years are uncomfortable with swallowing capsules or tablets. A liquid suspension of a drug that is orally delivered is the most desirable formulation for use in treating children, and also for use in treating older or sick people with difficulty swallowing capsules and tablets. An oral suspension offers users the flexibility and accuracy of dosing, with a palatable alternative to solid dosage forms of a drug, thus improving compliance and the medical outcome of a treatment.

Celebrex® or celecoxib (4-[5-(4-methylphenyl)-3-(trifluoromethyl)-1H-pyrazol-1-yl]benzenesulfonamide) is used to treat the signs and symptoms of juvenile idiopathic arthritis (JIA) in children aged 2-17 years. JIA is the most common form of arthritis in children and adolescents. Pain is the most common and distressing symptom of JIA and seems to be more frequent and intense compared with other rheumatic diseases. Most children with JIA are treated first with NSAIDs, including Celebrex® capsules for pain relief. Celebrex® is a very important treatment option for children with JIA.

Oral suspensions of celecoxib have been described, for instance in EP 1414409 B1, as stabilized by the addition of highly dispersed silicon dioxide and small amounts of polysorbate 80. Two such investigational suspensions (10 mg/ml and 20 mg/ml of celecoxib) were used in a clinical study to obtain approval of celecoxib for the treatment of JIA (Pfizer 2006, Briefing Document Celecoxib for JRA (NDA 20-998/S-021)).

However, the oral suspensions used in the above trial were not the formulations marketed by Pfizer (Pfizer 2006, Briefing Document Celecoxib for JRA (NDA 20-998/S-021)). The sponsor had technical problems producing these liquid suspensions on a large scale. Pfizer proposed discontinuing the development of the oral suspension formulation and using the capsule formulation for patients with JIA, although oral suspension formulation supports more accurate dosing for pediatric patients. Children 2 years or older (10 kg to ≦25 kg) with JIA currently are administered 50 mg capsules of celecoxib twice a day, and children that are >25 kg are administered 100 mg capsules of celecoxib twice a day. It is recommended that children who have difficulty in swallowing capsules should empty the contents of a capsule and sprinkle it on applesauce for dosing.

The current capsule dosing scheme in children with JIA is not only inconvenient, but also results in poor dosing accuracy, recommends higher doses of celecoxib for the smaller weight patients, and poses undue risk of increased adverse events (Pfizer 2006, Briefing Document Celecoxib for JRA (NDA 20-998/S-021)). Manual splitting of capsules can result in poor dosing accuracy. Lack of accuracy and flexibility in dosing, as well as administration problems, can result in poor compliance and poor treatment outcome of this debilitating disease in children. A liquid suspension of celecoxib is urgently needed for JIA patients.

Allopurinol [4-hydroxypyrazolo (3,4-d)-pyrimidine] is used in children in the treatment or prevention of abnormally high levels of uric acid in blood. Hyperuricemia in children is caused by many factors, including various neoplastic diseases, several cancer medication treatments, genetics and obesity. Hyperuricemia can lead to painful gouty arthritis, kidney disease and kidney failure. Allopurinol comes as a tablet to be taken by mouth, usually once or twice a day. A liquid suspension of allopurinol is urgently needed for children who cannot swallow allopurinol tablets.

Accordingly, it would be extremely useful to find compositions that form good aqueous suspensions of celecoxib and allopurinol, that have a useful shelf life and are easily re-suspended if settling occurs. Additionally, the drug suspensions should be easy to pour, pleasant in appearance and taste, stable for an extended period of time, and free of microbial contamination.

### BRIEF SUMMARY OF THE INVENTION

This present invention relates to the surprising finding that a combination of glycerol and insoluble cross-linked polyvinylpyrrolidone (crospovidone) is synergistic and forms good stable aqueous suspensions of either celecoxib or allopurinol, has a useful shelf life and is easily re-suspended if settling occurs. It was observed that glycerol alone or crospovidone alone does not prevent the settling and/or caking of the drug.

The invention is further surprising in that glycerol, in combination with crospovidone, is the only sugar alcohol among a range of sugar alcohols that were tested which yielded positive results. Other sugar alcohols, such as sorbitol and xylitol, in combination with crospovidone did not prevent the setting and/or caking of celecoxib and allopurinol. Similarly, insoluble cross-linked polyvinylpyrrolidone (crospovidone) in combination with glycerol prevented settling and/or caking of celecoxib and allopurinol. In contrast, soluble polyvinylpyrrolidone did not yield positive results.

These observations can lead to an aqueous pharmaceutical suspension that is stable over a long period of time. This suspension is comprised of an aqueous buffer system, wetting agent(s), xanthan gum, crospovidone, glycerol and taste modifying agents selected from the group consisting of bulk sweeteners, flavoring agents and mixtures thereof.

Accordingly, in one embodiment of the present invention, there is provided a stable aqueous pharmaceutical oral suspension comprising a pharmaceutically acceptable form of celecoxib, an aqueous buffer system, xanthan gum, crospovidone, and glycerol.

The present invention provides a method of treatment with a composition of celecoxib, as described above, for treating a medical condition or disorder in a mammal where treatment with celecoxib is indicated. Said composition to be orally administered to the mammal once or twice a day.

And, in another embodiment, there is provided a stable oral suspension comprising a pharmaceutically acceptable form of allopurinol, an aqueous buffer system, xanthan gum, crospovidone, and glycerol.

The present invention provides a method of treatment with a composition of allopurinol, as described above, for treating a medical condition or disorder in a mammal, where treatment with allopurinol is indicated. Said composition to be orally administered to the mammal in single or divided doses per day.

Accordingly, in one embodiment, there is an aqueous pharmaceutical liquid suspension for oral administration to a mammal in need thereof comprising:
a) a pharmaceutically acceptable salt selected from the group consisting of celecoxib and allopurinol;
b) crospovidone;
c) glycerol; and
d) at least one of each of the group consisting of an aqueous buffer system, a suspending agent, a wetting agent, a sweetening agent, a preservative, and a pharmaceutically acceptable liquid carrier.

### DETAILED DESCRIPTION OF THE INVENTION

The terms “about” and “essentially” mean±10 percent.

As used herein “percent w/v” refers to the percent weight of the total composition.

The term “comprising” is not intended to limit inventions to only claiming the present invention with such comprising language. Any invention using the term “comprising” could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

As used herein, the term “mammal” is defined as any class of warm-blooded higher vertebrates that includes humans.

For purposes of this invention, a suspension means a liquid with solid particles dispersed substantially throughout the system. The properties of a liquid suspension, according to the invention, are greatly influenced by the particle size of the suspended active substance. As used herein, a “particle” may be a crystal, a granule, agglomerate, or any un-dissolved solid material. To achieve the rapid onset of activity, which is desirable, a small particle size is essential, ensuring the fastest possible dissolution of the active substance in the gastrointestinal tract. The particle size distribution in suspension is also a very important factor characterizing the physical stability (for example, sedimentation ratio, etc.) of the formulation. Generally, as the particle size becomes smaller, the sedimentation ratio increases, and the physical stability is improved. Air-jet milling, ball milling, mortar milling, micronization or any other method known in the art for decreasing particle size may achieve the active substance's particle size.

In one embodiment, the present invention provides for a stable oral suspension, which includes celecoxib or a pharmaceutically acceptable salt thereof. The phrase “pharmaceutically acceptable salts” as used herein, includes salts commonly used to form alkali metal salts and addition salts of free acids or free bases. Suitable pharmaceutically acceptable acid addition salts of celecoxib may be prepared from an inorganic acid or from an organic acid.

Preferably, the particle size distribution of the celecoxib particles in suspension is greater than about 3 micron to less than about 50 micron. Even more preferably, the particle size spectrum of celecoxib, that is suitable for suspension according to the invention, includes at least 90% of the particles that are smaller than 50 micron, preferably at least 50% of the particles are smaller than 10 micron, and most preferably about 90% of the particles are smaller than 10 micron. Particle diameter distributions may be determined by laser diffraction methods.

The dosage amounts of celecoxib present in the liquid compositions may vary dependent upon patient needs, but preferably celecoxib is present in the liquid at about 5 to 30 mg/mL (0.5 to 3.0% w/v) and more preferably at about 7.5 to 20 mg/mL (0.75 to 2.0% w/v). Most preferably, the celecoxib is present in the liquid at about 10 mg/ml (1% w/v).

As used herein, a “unit dose volume” of the aqueous suspension is a convenient volume for dosing the product to a recipient. The dosing directions instruct the recipient to take amounts that are multiples of the unit doses, depending on, for example, the age or weight of the recipient. Typically the unit dose volume of the suspension will contain an amount of celecoxib that is therapeutically effective for the smallest patient. The suspension can be dispensed from a suspension dispenser.

The liquid formulations may be used to treat any disease indication for which celecoxib may be prescribed in a mammal, irrespective of age. Preferably, the compositions are for use in the treatment of children, most preferably treatment of children aged 2 to 11 years. The liquid suspension may also be used in treatment of animals, as it is convenient and dosage can be accurately controlled.

Exemplary disease indications include; for relief of the signs and symptoms of osteoarthritis, for relief of the signs and symptoms of rheumatoid arthritis in adults, for relief of the signs and symptoms of juvenile rheumatoid arthritis in patients two years and older, for the relief of signs and symptoms of ankylosing spondylitis, for the management of acute pain in adults, for the treatment of primary dysmenorrhea, and to reduce the number of adenomatous colorectal polyps in familial adenomatous polyposis, as an adjunct to usual care (e.g., endoscopic surveillance, surgery).

In another embodiment, the present invention provides for a stable oral suspension, which includes allopurinol or a pharmaceutically acceptable salt thereof. In the context of the present invention, the term “allopurinol” refers also to the different tautomers of the compound, since it is a tautomeric mixture of 1H-pyrazolo[3,4-d]pyrimidin-4-ol and 1,5-dihydro-4H-pyrazolo[3,4-d]pyrimidin-4-one]].

Preferably, the particle size distribution of the allopurinol particles in suspension is greater than about 3 micron to less than about 50 micron. Even more preferably, particle size spectrum of allopurinol that is suitable for a suspension according to the invention includes at least 90% of the particles that are smaller than 50 micron, preferably at least 50% of the particles are smaller than 10 micron, and most preferably about 90% of the particles are smaller than 10 micron. Particle diameter distributions may be determined by laser diffraction methods.

The dosage amounts of allopurinol present in the liquid compositions may vary dependent upon patient needs, but preferably allopurinol is present in the liquid at about 10 to 30 mg/mL (1.0 to 3.0% w/v) and more preferably at about 15 to 25 mg/mL (1.5 to 2.5% w/v). Most preferably, the allopurinol is present in the liquid at about 20 mg/mL (1% w/v).

As used herein a “unit dose volume” of the aqueous suspension is a convenient volume for dosing the product to a recipient. The dosing directions instruct the recipient to take amounts that are multiples of the unit doses depending on, for example, the age or weight of the recipient. Typically, the unit dose volume of the suspension will contain an amount of allopurinol that is therapeutically effective for the smallest patient. The allopurinol suspension can be dispensed from a suspension dispenser.

The liquid formulations may be used to treat any indication for which allopurinol may be prescribed in a mammal, irrespective of age. Preferably, the liquid allopurinol compositions are for use in the treatment of children, most preferably in children aged 2 to 8 years. The liquid suspension may also be used in animals, as it is convenient and dosage can be accurately controlled. Allopurinol reduces serum and urinary uric acid concentrations. Its use should be individualized by a physician for each patient and requires an understanding of its mode of action and pharmacokinetics. A few examples of indications for allopurinol are recited in the following section.

Allopurinol is indicated in the management of patients with signs and symptoms of primary or secondary gout (acute attacks, tophi, joint destruction, uric acid lithiasis, and/or nephropathy). It is also indicated in the management of patients with leukemia, lymphoma, and malignancies who are receiving cancer therapy, which causes elevations of serum and urinary uric acid levels. Treatment with allopurinol should be discontinued when the potential for overproduction of uric acid is no longer present. Another indication for allopurinol is for the management of patients with recurrent calcium oxalate calculi whose daily uric acid excretion exceeds 800 mg/day in male patients and 750 mg/day in female patients. Therapy in such patients should be carefully assessed initially and reassessed periodically to determine, in each case, that treatment is beneficial and that the benefits outweigh the risks.

In another embodiment, the present invention relates the surprising finding that a combination of glycerol and insoluble cross-linked polyvinylpyrrolidone (crospovidone) is synergistic and forms good stable aqueous suspensions of celecoxib and allopurinol, has a useful shelf life and is easily re-suspended if settling occurs. “Good stable aqueous suspension” as used to describe a suspension means that (a) drug particles remain suspended in the suspension vehicle such that dose uniformity is obtainable, and/or (b) the suspension exhibits substantially uniform drug particle dispersion and substantially no phase separation during the stationary room temperature storage period of at least one week after preparation.

The glycerol can be present in an amount of from about 0.5 to about 50% w/v of the suspension, preferably from about 1 to about 10% w/v of the suspension and most preferably in an amount of about 5% w/v of the suspension.

Polyvinylpyrrolidone (PVP), also commonly called polyvidone or povidone, is a water-soluble polymer made from the monomer N-vinylpyrrolidone.

Polyvinylpolypyrrolidone (polyvinylpolypyrrolidone, PVPP, crospovidone, crospolividone or E1202) is a highly cross-linked modification of polyvinylpyrrolidone (PVP). Unlike, PVP cross-linked crospovidone, it is water insoluble and, in the present invention, forms good stable aqueous suspensions of celecoxib and allopurinol with an excipient base comprising glycerol.

Crospovidone can be present in an amount of from about 0.5 to about 30% w/v of the suspension, preferably from about 1% to about 10% w/v of the suspension and most preferably in an amount of about 5% w/v of the suspension.

Preferably, the particle size of the crospovidone particles used in this invention is from about 3 micron to about 150 micron, more preferably, from about 3 micron to about 40 micron and most preferably a micronized grade of crospovidone from about 3 micron to about 10 micron. For example, a micronized grade crospovidone designated Kollidone CL-M is available from BASF.

Suspending agents, which may be used according to the present invention, include, but are not limited to, xanthan gum, guar gum and microcrystalline cellulose. Preferably, the suspending agent is xanthan gum. Xanthan gum is a high molecular weight natural carbohydrate, specifically a polysaccharide. The xanthan gum can be present in an amount from about 0.10 to about 1% w/v of the suspension, preferably from about 0.20 to 0.50% w/v and most preferably in an amount of about 0.4% w/v of the suspension.

One or more wetting agents are present in suspension compositions of the invention. Surfactants, including nonionic, anionic, cationic and zwitterionic surfactants, are preferred wetting agents in suspension compositions of the invention. Non-limiting examples of surfactants that can be used as wetting agents in compositions of the invention include polyethylene glycols (PEGs), sorbitan monolaurate, polysorbate 80, polysorbate 20, sodium lauryl sulfate and the like. Several grades of PEGs can be employed at concentrations including those having average molecular weights of from about 400 to 4000. PEG 4000 is particularly preferred because higher molecular weights develop high viscosities and detract from taste.

An embodiment of the invention comprises a polysorbate at a concentration of 0.01% to 0.20% weight/volume (w/v) and a polyethylene glycol at a concentration of 0.1% to 2.0% w/v which composition forms a good stable flocculated suspension in water.

Sweeteners, which may be used according to the present invention, may be any natural or artificial sweetener. In terms of natural sweeteners, these include, but are not limited to, glucose, fructose, invert sugar, sorbitol, sucrose, maltose, xylose, ribose, mannose, corn syrup solids, xylitol, mannitol, maltodextrins, and mixtures thereof. In one embodiment, the natural sweetener is xylitol.

In terms of artificial sweeteners, these include, but are not limited to, saccharin, aspartame and sucralose. In one embodiment, the artificial sweetener is aspartame.

Preservatives, which may be used according to the present invention, include, but are not limited to, benzoic acid, sodium benzoate, potassium sorbate, cresol, cetrimide, citric acid and sodium citrate, and alkyl hydroxybenzoates (parabens). Preferably, the preservative is selected from an alkyl hydroxybenzoate, such as methyl hydroxybenzoate, ethyl hydroxybenzoate, propyl hydroxybenzoate (as base or sodium salt) or a combination thereof.

Buffers which may be used, according to the present invention, include suitable buffers that are not chemically reactive with the other ingredients, and which may be present in an amount sufficient to provide the desired degree of pH buffering. In this regard, a buffer system comprising of an aqueous mixture of an acid, wherein the acid is phosphoric, succinic, tartaric, lactic, or citric acid, and a base, wherein the base is trisodium citrate dehydrate, sodium hydroxide, or disodium hydrogen phosphate, is for maintaining the pH in the range from 4 to 6. Buffer citric acid and trisodium citrate dihydrate at 0.10% and 0.30% w/v, respectively are preferred.

Flavors incorporated in the composition may be chosen from synthetic flavor oils and flavoring aromatics and/or natural oils, extracts from plant leaves, flowers, fruits, and so forth and combinations thereof. Also useful as flavors are vanilla, citrus oils, including lemon, orange, lime and grapefruit, and fruit essence, including apple, grape, banana, pear, peach, strawberry, raspberry, cherry, plum, pineapple, apricot, and so forth.

The carrier/vehicle used in the compositions of the invention is preferably water, although other suitable water-containing (aqueous) carriers/vehicles known to the skilled person may also be used.

The following examples are intended for illustration only and are not intended to limit the scope of the invention in any way.

### EXAMPLES

### Example 1—Test Methods

Sedimentation Ratio:

Suspensions were shaken well and poured into 10 ml graduated cylinders, sealed tightly and kept undisturbed at room temperature in the dark. The sedimentation volume and nature of the separated phase were noted at various time intervals. Sedimentation ratio F is the ratio of the equilibrium volume of the sediment Vₛ to the total volume of the suspension V₀ and is derived from the following equation F=Vₛ/V₀.

The value of F normally ranges from 0 to 1. Suspension with F value 1 is the ideal suspension. The suspension is flocculated, and there is no sedimentation or caking. This suspension is also visibly appealing since there is no visible clear supernatant. This type of suspension is said to be in flocculation equilibrium. If the sedimentation volume is 0.5, the loose flecks of the sediment occupy 50% volume of the suspension. The F value of a deflocculated suspension is relatively small, 0.2 or lower.

Settling and Caking of Suspensions:

Settling and aggregation may result in formation of cakes that are difficult to re-suspend. Suspensions were shaken well and stored in 15 ml conical tubes, sealed tightly and kept undisturbed at room temperature in the dark. Re-dispersion of a suspension was determined at various time intervals. Suspensions were held upright between the fingers and rotated clockwise upside down through 180° in a semi-circle path and back in the counterclockwise direction. This process was repeated continuously for five minutes. Both celecoxib and allopurinol are insoluble in water and form cakes in water. The amount of caking of each suspension is determined by comparing it with a suspension of the drug in water by the following equation. Percent Caking/Settling=(Volume of Cake in Suspension/Volume of Cake in Water)×100.

Particle Size Distribution and Zeta Potential Measurements were performed using a Malvern Instruments™ Zetasizer Nano ZS Dynamic Light Scattering system (DLS) with disposable plastic cuvettes and a Zeta cell. DLS is a commonly used term to describe a technique which measures the particle size and estimated distribution of submicron particulate systems. To measure zeta potential, an electric field is applied to a dispersion of particles, which then move with a velocity related to their zeta potential. This enables the calculation of electrophoretic mobility and, from this, the zeta potential and zeta potential distribution.

Viscosity Measurements.

Rheological consideration of a suspension is of great importance for the stability because viscosity can modify the sedimentation rates. Maintaining the proper viscosity of suspensions is also important to ensure the accuracy of dosing and ease of application. Apparent viscosity @ 125 sec⁻¹ and yield stress for suspensions at 25° C. was performed on a TA Instruments™ AR-G2 Rheometer using 40 mm parallel plate geometry. The sample volume used is a few milliliters per sample. The yield stress is the applied stress we must exceed in order to make a suspension flow. Approximate yield stress measurements were obtained by plotting the shear stress values for a range of shear rates, fitting a curve to the data, and extrapolating through the stress axis; intersect on the stress axis gives the yield stress. The pascal (Pa) is the unit of pressure or stress in the International System of Units (SI). A centipoise (cP) is a non-SI measurement unit of dynamic viscosity in the centimeter gram second system of units.

### Example 2

A suspension formulation celecoxib suspension C-1 was prepared comprising components shown in Table 1 in the following manner. In a water bath, 100 ml of water was heated to 70° C. and 300 mg methylparaben and 40 mg propylparaben were added to the heated water. The solution was stirred with an overhead polytron stirrer at about 300 rpm. After the parabens were completely dissolved, 60 g xylitol was added with continuous stirring. A clear solution was cooled to room temperature and the following components were added with stirring: 200 mg of anhydrous citric acid, 600 mg sodium citrate dihydrate, 2 g NaCl, 1 g polyethylene glycol (PEG) 4000 and 0.1 g polysorbate 80. After all the ingredients were dissolved, 10 g of crospovidone was slowly added into the mixture with stirring. Celecoxib, 2 g was added to the mix and stirred for 30 minutes to get a uniform suspension. Xanthan gum, 600 mg was separately dispersed in 10 g of glycerol and slowly added to the suspension with stirring. Finally, coloring and flavoring were added and the volume was adjusted to 200 ml with water. Celecoxib suspensions C-2 (no glycerol) and C-3 (no crospovidone) were prepared by mixing components shown in Table 1 as described for celecoxib suspension C-1 above.

TABLE 1
Celecoxib Suspensions: Glycerol, and Crospovidone Form Good
Aqueous Suspensions

| | Percent W/V | | |
|---|---|---|---|
| Component | C-1 | C-2 | C-3 |
| Celecoxib | 1 | 1 | 1 |
| Xanthan gum | 0.3 | 03 | 0.3 |
| Xylitol | 30.0 | 30.0 | 30.0 |
| Glycerol | 5 | 0 | 5 |
| Crospovidone | 5 | 5 | 0 |
| PEG 4000 | 0.5 | 0.5 | 0.5 |
| Polysorbate 80 | 0.05 | 0.05 | 0.05 |
| Citric Acid | 0.1 | 0.1 | 0.1 |
| Trisodium citrate | 0.3 | 0.3 | 0.3 |
| dihydrate | | | |
| Methylparaben | 0.15 | 0.15 | 0.15 |
| Propylparaben | 0.02 | 0.02 | 0.02 |
| Flavoring | 0.1 | 0.1 | 0.1 |
| Coloring | 0.05 | 0.05 | 0.05 |
| NaCl | 1.0 | 1.0 | 1.0 |

The effect of combining crospovidone and glycerol on a celecoxib suspension was evaluated by measuring the sedimentation ratio, particle size distributions, and zeta potential of celecoxib suspensions C-1, C-2 and C-3. Sedimentation ratio, particle size distribution and zeta potential of a suspension with glycerol and crospovidone (celecoxib suspension C-1) were compared with a suspension with crospovidone alone (celecoxib suspension C-2) or glycerol alone (celecoxib suspension C-3). Results are shown in Table 2.

TABLE 2
Celecoxib Suspensions: Glycerol, and Crospovidone Form Good
Aqueous Suspensions

| | Day 7 | Day 14 | Particle Size | |
|---|---|---|---|---|
| | Sedimentation | Sedimentation | Distribution, | Zeta |
| Composition | Ratio, F^{a} | Ratio, F | micron | Potential |
| C-1 | 1.00 | 1.0 | 9.0 | −7.27 |
| Glycerol + | | | | |
| Crospovidone | | | | |
| C-2 | 0.62 | 0.20 | 10.5 | −4.76 |
| No Glycerol | | | | |
| C-3 | 0.20 | 0.10 | 41.9 | −10.4 |
| No | | | | |
| Crospovidone | | | | |

^{a}Suspension with F value 1 is the ideal suspension.

Celecoxib suspension C-1 with both glycerol and crospovidone is an ideal suspension, as no sedimentation or caking was observed after 14 days (Table 2). Particle size distribution of C-1 celecoxib suspension was less than 10 microns and zeta potential was −7.27. The combination of glycerol and crospovidone is synergistic, as it prevents the sedimentation of the celecoxib suspension, whereas glycerol alone or crospovidone alone does not prevent the settling and/or caking of the drug. Celecoxib suspensions without glycerol (celecoxib suspension C-2) or crospovidone (celecoxib suspension C-3) showed a high degree of sedimentation. Celecoxib suspension C-2 had 80% sedimentation (F=0.2) and celecoxib suspension C-3 had about 90% sedimentation (F=0.1) after 14 days (Table 2). Particle size distributions for celecoxib suspension C-2 and celecoxib suspension C-3 were also substantially higher than 10 microns.

This data demonstrates that the presence of both glycerol and crospovidone is synergistic and greatly increases anti-sedimentation properties of celecoxib suspensions compared to suspensions comprising either glycerol or crospovidone alone. This synergistic anti-sedimentation effect indicates an increase in physical stability of the celecoxib suspension.

### Example 3

To further evaluate the effect of glycerol on a celecoxib suspension, four additional formulations (celecoxib suspensions C-4, C-5, C-6, and C-7) were prepared by mixing components as described earlier for the preparation of C-1. These formulations comprise the same components as celecoxib suspension C-1 (Table 1) except 1% (celecoxib suspension C-4), 2% (celecoxib suspension C-5), or 10% (celecoxib suspension C-6) glycerol instead of 5% glycerol (celecoxib suspension C-1). A formulation suspension C-7 was prepared containing same components as celecoxib suspension C-1 (Table 1) except glycerol was replaced with 5% sorbitol.

The effect of varying amounts of glycerol on sedimentation ratio, particle size distributions, and zeta potential on a celecoxib suspension is shown in Table 3. Addition of 1%, 2% and 5% of glycerol to a celecoxib suspension prevents sedimentation. About 10% of sedimentation (F=0.9) of a celecoxib suspension was observed when 10% of glycerol was added to the suspension (Table 3). This sedimentation was accompanied with a substantial increase in particle size distribution to 33.5 micron.

TABLE 3
Celecoxib Suspensions: Effect of Glycerol

| | Day 7 | Day 14 | Particle Size | |
|---|---|---|---|---|
| | Sedimentation | Sedimentation | Distribution, | Zeta |
| Composition | Ratio, F^{a} | Ratio, F | micron | Potential |
| C-2 | 0.62 | 0.20 | 10.5 | −4.76 |
| No Glycerol | | | | |
| C-4 | 1.00 | 0.92 | 10.3 | −4.95 |
| 1% Glycerol | | | | |
| C-5, | 1.00 | 0.96 | 9.0 | −7.31 |
| 2% Glycerol | | | | |
| C-1 | 1.00 | 1.00 | 9.0 | −7.27 |
| 5% Glycerol | | | | |
| C-6 | 0.90 | 0.90 | 33.5 | −4.60 |
| 10% Glycerol | | | | |

^{a}Suspension with F value 1 is the ideal suspension.

### Example 4

This example compares the anti-sedimentation properties of different sugar alcohols on celecoxib suspensions (Table 4). As discussed earlier, a celecoxib suspension C-2 with xylitol only and with no added glycerol, showed a high degree of sedimentation and addition of 5% glycerol prevented the sedimentation of a celecoxib suspension C-1. In contrast, addition of 5% sorbitol (celecoxib suspension C-7) to a suspension did not prevent sedimentation (F=0.36) after 14 days (Table 4). Therefore, glycerol is the only sugar alcohol tested that, in combination with crospovidone, prevents sedimentation of celecoxib suspension.

TABLE 4
Celecoxib Suspensions: Effect of Glycerol

| | Day 7 | Day 14 | Particle Size | |
|---|---|---|---|---|
| | Sedimentation | Sedimentation | Distribution, | Zeta |
| Composition | Ratio, F^{a} | Ratio, F^{a} | micron | Potential |
| C-2 | 0.62 | 0.20 | 10.5 | −4.76 |
| Xylitol Only | | | | |
| C-1 | 1.00 | 1.00 | 9.0 | −7.27 |
| Xylitol + 5% | | | | |
| Glycerol | | | | |
| C-7 | 0.70 | 0.36 | 15.3 | −3.39 |
| Xylitol + 5% | | | | |
| Sorbitol | | | | |

^{a}Suspension with F value 1 is the ideal suspension.

### Example 5

To evaluate the effects of wetting agents PEG-4000 and polysorbate 80 on celecoxib suspension, two formulations (celecoxib suspensions C-8, C-9) were prepared by mixing components as described earlier for the preparation of celecoxib suspension C-1. These formulations comprise the same components as celecoxib suspension C-1 (Table 1) except formulation celecoxib suspension C-8 was prepared without PEG-4000 and formulation celecoxib suspension C-9 was prepared without polysorbate 80.

Particle size distribution of C-1 celecoxib suspension with both the wetting agents PEG-4000 and polysorbate 80 was less than 10 microns and zeta potential was −7.27 (Table 5). In contrast, particle size distribution of a celecoxib suspension without PEG-4000 (celecoxib suspension C-8) or polysorbate 80 (celecoxib suspension C-9) was substantially higher (41.9 and 65.1 micron, respectively) than celecoxib suspension C-1. This data shows that the presence of both the wetting agents lowers the particle size distribution of celecoxib suspension.

TABLE 5
Celecoxib Suspensions: Effect of Wetting Agents PEG-4000 and
Polysorbate 80

| | Day 7 | Day 14 | Particle Size | |
|---|---|---|---|---|
| | Sedimentation | Sedimentation | Distribution, | Zeta |
| Composition | Ratio, F^{a} | Ratio, F^{a} | micron | Potential |
| C-1 | 1.00 | 1.00 | 9.0 | −7.27 |
| Complete | | | | |
| C-8 | 1.00 | 0.96 | 41.9 | −4.99 |
| No PEG 4000 | | | | |
| C-9 | 1.00 | 0.96 | 65.1 | −7.31 |
| No Polysorbate | | | | |

^{a}Suspension with F value 1 is the ideal suspension

### Example 6

To evaluate the effect of suspending agent xanthan gum on celecoxib suspension, another formulation (celecoxib suspension C-10) with 0.50% xanthan gum instead of 0.3% xanthan gum (celecoxib suspension C-1) was prepared by mixing components, as described earlier for C-1.

The results in Table 6 show that particle size distribution of celecoxib suspension was lowered from 9.0 micron to 5.8 micron with an increase of xanthan gum from 0.3% (celecoxib suspension C-1) to 0.5% (celecoxib suspension C-10). The decrease in particle size distribution was accompanied by an improvement in zeta potential from −7.27 to −18.20 (Table 6).

As shown in Table 6, both yield stress value and apparent viscosity increased with an increase of xanthan gum from 0.3% (celecoxib suspension C-1) to 0.5% (celecoxib suspension C-10). This increase in yield value indicates the presence of relatively high flow resistance at low stresses, for example gravitational stresses involved in particle sedimentation. Despite high yield values exhibited by celecoxib, the apparent viscosities of suspensions C-1 and C-10 were still relatively low, indicating good fluidity of the suspensions after yield value was reached. This data indicates that the celecoxib suspensions C-1 and C-10 are thick at rest, but fluid after moderate agitation. This increased thickness at rest will result in increased physical stability.

TABLE 6
Celecoxib Suspensions: Effect of Xanthan Gum

| | | | | | Apparent |
|---|---|---|---|---|---|
| | Day 14 | Particle Size | | Yield | Viscosity |
| | Sedimentation | Distribution, | Zeta | Stress, | @ 125 |
| Composition | Ratio, F^{a} | micron | Potential | Pa | S⁻¹, cP |
| C-1 | 1.00 | 9.0 | −7.27 | 2.96 | 71.8 |
| 0.30% | | | | | |
| Xanthan | | | | | |
| Gum | | | | | |
| C-10 | 1.00 | 5.8 | −18.20 | 6.91 | 133 |
| 0.50% | | | | | |
| Xanthan | | | | | |
| Gum | | | | | |

^{a}Suspension with F value 1 is the ideal suspension

### Example 7

To study the effect of combining crospovidone and glycerol on an allopurinol suspension, a formulation allopurinol suspension C-11 was prepared, comprising components shown in Table 7 in the following manner. In a water bath, 100 ml of water was heated to 70° C. and 300 mg methylparaben and 40 mg propylparaben were added to the heated water. The solution was stirred with an overhead polytron stirrer at about 300 rpm. After the parabens were completely dissolved, 60 g xylitol was added with continuous stirring. The clear solution was cooled to room temperature and the following components were added with stirring: 200 mg of anhydrous citric acid, 600 mg sodium citrate dihydrate, 2 g NaCl, 1 g polyethylene glycol (PEG) 4000 and 0.1 g polysorbate 80. After all the ingredients were dissolved, 10 g of crospovidone was slowly added in the mixture with stirring. Allopurinol 4 g was added to the mix and stirred for 30 minutes to get a uniform suspension. Xanthan gum, 600 mg was separately dispersed in 10 g of glycerol and slowly added to the suspension with stirring. Finally, coloring and flavoring were added and the volume was adjusted to 200 ml with water. Two additional suspension formulations without glycerol (allopurinol suspension C-12) and without crospovidone (allopurinol suspension C-13) were prepared by mixing components shown in Table 7 as described earlier.

TABLE 7
Allopurinol Suspensions: Glycerol, and Crospovidone Form Good
Aqueous Suspensions

| | Percent W/V | | |
|---|---|---|---|
| Component | C-1 | C-2 | C-3 |
| Allopurinol | 2 | 2 | 2 |
| Xanthan gum | 0.3 | 0.3 | 0.3 |
| Xylitol | 30.0 | 30.0 | 30.0 |
| Glycerol | 5 | 0 | 5 |
| Crospovidone | 5 | 5 | 0 |
| PEG 4000 | 0.5 | 0.5 | 0.5 |
| Polysorbate 80 | 0.05 | 0.05 | 0.05 |
| Citric Acid | 0.1 | 0.1 | 0.1 |
| Trisodium citrate | 0.3 | 0.3 | 0.3 |
| dihydrate | | | |
| Methylparaben | 0.15 | 0.15 | 0.15 |
| Propylparaben | 0.02 | 0.02 | 0.02 |
| Flavoring | 0.1 | 0.1 | 0.1 |
| Coloring | 0.05 | 0.05 | 0.05 |
| NaCl | 1.0 | 1.0 | 1.0 |

The effect of combining crospovidone and glycerol in an allopurinol suspension was evaluated by measuring the percent of caking or settling of the drug, particle size distributions, and zeta potential of suspensions C-11, C-12 and C-13. Allopurinol suspensions did not show a phase separation so the sedimentation ratio was not measured. Results are shown in Table 8.

Sedimentation or caking of the drug was not observed after 14 days in Allopurinol suspension C-11, which contained both glycerol and crospovidone (Table 8). Particle size distribution of allopurinol suspension C-11 suspension was less than 10 microns and zeta potential was −4.62 (Table 8). The combination of glycerol and crospovidone is synergistic as it prevents the settling or caking of the drug in an allopurinol suspension, whereas glycerol alone or crospovidone alone does not prevent the settling and/or caking of the drug. Allopurinol suspensions without glycerol (allopurinol suspension C-12) or crospovidone (allopurinol suspension C-13) showed a high degree of sedimentation. C-12 had 92% settling or caking of the drug and allopurinol suspension C-13 had about 97% settling or caking of the drug after 14 days (Table 8). Particle size distributions for allopurinol suspension C-12 and allopurinol suspension C-13 were also substantially higher than 10 microns (Table 8).

TABLE 8
Allopurinol Suspensions: Glycerol, and Crospovidone Form Good
Aqueous Suspensions

| | Day 7 | Day 14 | | |
|---|---|---|---|---|
| | % Drug | % Drug | Particle Size | |
| | Settling/ | Settling/ | Distribution, | Zeta |
| Composition | Caking | Caking | micron | Potential |
| C-11 | None | None | 5.9 | −4.62 |
| Glycerol + Crospovidone | | | | |
| C-12 | 18 | 92 | 12.4 | −1.56 |
| No Glycerol | | | | |
| C-13 | 23 | 97 | 44.4 | −22.2 |
| No Crospovidone | | | | |

This data shows that the presence of both glycerol and crospovidone is synergistic and greatly increases anti-caking properties of allopurinol suspensions compared to suspensions comprising either glycerol or crospovidone alone. This synergistic anti-caking effect indicates an increase in physical stability of the allopurinol suspension.

### Example 8

To further evaluate the effect of crospovidone on an allopurinol suspension, four additional formulations (allopurinol suspensions C-14, C-15, C-16, and C-17) were prepared by mixing components as described earlier for the preparation of allopurinol suspension C-11. These formulations comprise the same components as allopurinol suspension C-11 (Table 7) except 1% (allopurinol suspension C-14), 2% (allopurinol suspension C-15), or 10% (allopurinol suspension C-16), crospovidone instead of 5% crospovidone (C-11). A formulation suspension C-17 was prepared containing the same components as allopurinol suspension C-11 (Table 7), except water insoluble crospovidone was replaced with 5% soluble povidone.

The effect of varying amounts of crospovidone on drug settling or caking, particle size distributions, and zeta potential on an allopurinol suspension is shown in Table 9. Addition of 5% and 10% of crospovidone to an allopurinol suspension completely prevents drug settling or caking after 14 days. Addition of 5% and 10% of crospovidone also substantially reduced particle size distribution and increased the zeta potential compared to a suspension without crospovidone (Table 9).

TABLE 9
Allopurinol Suspensions: Effect of Crospovidone

| | Day 7 | Day 14 | | |
|---|---|---|---|---|
| | Drug | Drug | Particle Size | |
| | Settling/ | Settling/ | Distribution, | Zeta |
| Composition | Caking % | Caking % | micron | Potential |
| C-13 | 20 | 100 | 44.40 | −22.2 |
| No Crospovidone | | | | |
| C-14, 1% CP | 10 | 15 | 27.90 | −8.31 |
| C-15, 2% CP | None | 17 | 9.13 | −5.01 |
| C-11, 5% CP | None | None | 5.94 | −4.62 |
| C-16, 10% CP | None | None | 6.23 | −3.72 |

### Example 9

This example compares the effects of crospovidone and povidone on an allopurinol suspension (Table 10). A formulation suspension C-17 comprises 5% soluble povidone instead of 5% cross linked povidone (crospovidone). An allopurinol suspension (C-11) with crospovidone and glycerol prevents the settling or caking of drug in an allopurinol suspension after 14 days and lowered the particle size distribution as compared to a suspension lacking crospovidone (Table 10). In contrast, inclusion of povidone and glycerol in an allopurinol suspension (C-17) does not prevent settling and caking of the drug and does not reduce the particle size distributions compared to a suspension lacking povidone (Table 10).

TABLE 10
Allopurinol Suspensions: Effect of Crospovidone and Povidone

| | Day 7 | Day 14 | | |
|---|---|---|---|---|
| | Drug | Drug | Particle Size | |
| | Settling/ | Settling/ | Distribution, | Zeta |
| Composition | Caking % | Caking % | micron | Potential |
| C-13 | 20 | 100 | 44.4 | −22.2 |
| No Povidone or | | | | |
| Crospovidone | | | | |
| C-17, Plus | 12 | 82 | 62.3 | −6.72 |
| Povidone | | | | |
| C-11, Plus | None | None | 5.9 | −4.62 |
| Crospovidone | | | | |

### Example 10

To evaluate the effect of suspending agent xanthan gum on an allopurinol suspension, another formulation (C-18) with 0.5% xanthan gum instead of 0.3% xanthan gum (allopurinol suspension C-11) was prepared by mixing components as described earlier for allopurinol suspension C-11.

Results in Table 11 show that particle size distribution of an allopurinol suspension was lowered from 5.90 micron to 4.10 micron with an increase of xanthan gum from 0.3% (allopurinol suspension C-1) to 0.5% (allopurinol suspension C-10). The decrease in particle size distribution was accompanied by an improvement in zeta potential from −4.62 to −17.80 (Table 11).

TABLE 11
Allopurinol Suspensions: Effect of Xanthan Gum

| | Day 7 | Day 14 | | |
|---|---|---|---|---|
| | Drug | Drug | Particle Size | |
| | Settling/ | Settling/ | Distribution, | Zeta |
| Composition | Caking % | Caking % | micron | Potential |
| C-11 | None | None | 5.9 | −4.62 |
| 0.30% Xanthan Gum | | | | |
| C-18 | None | None | 4.1 | −17.8 |
| 0.50% Xanthan Gum | | | | |

### Example 11

Storage Stability Test

The storage stability of the celecoxib suspension C-1 has been tested over a 6-month storage period. The celecoxib suspension C-1 was divided equally into three amber PVC bottles (30 ml volume). These bottles were stored at 25° C. At the end of the 6-month storage period the composition of celecoxib suspension C-1 was analyzed by HPLC chromatography and the amount of celecoxib and its known impurities were determined by a validated HPLC assay. An initial concentration of celecoxib in C-1 suspension was 9.90±0.10 mg/ml and after 6-month storage amount of celecoxib was not decreased and was found to be 10.0±0.19 mg/ml. No increase in impurities was observed during the 6-month storage period. Less than 5% sedimentation or phase separation was observed. Visibly no irreversible caking was observed during the 6-month storage period.

### Example 12

Comparison of pharmacokinetics (PK) of celecoxib after administration of a single dose of two oral liquid celecoxib suspensions and a Celebrex® (celecoxib) capsule in a human volunteer.

Two additional formulations, celecoxib suspension C-18 and C-19 were prepared by mixing components as described earlier for the preparation of C-1. Celecoxib liquid suspension C-18 comprises same components as the formulation C-1 (Table 1) except the concentration of xanthan gum was 0.40%. The formulation C-19 comprises the same components as celecoxib suspension C-1 (Table 1) except the concentration of xanthan gum was 0.45%, pH was 4.5, and it contained an additional excipient magnesium stearate (0.027%).

The relative bioavailability of celecoxib, administered as a liquid oral suspension C-18 or as a celecoxib suspension C-19 and as a Celebrex® capsule was determined in a healthy volunteer. On study day, the subject, after overnight fasting, received either one 200 mg Celebrex® capsule or 20 ml of liquid oral suspension formulation of celecoxib (10 mg/ml), C-18 or C-19 with 250 ml of water. Between each administration there was a washout period of two weeks. A pre-dose blood sample was taken prior to drug administration. Blood samples (3 ml) were collected at predefined time points as before drug administration (zero time) and at 0.5, 1, 2, 3, 4, 6, 8 and 29 h after dosing into single evacuated blood tubes containing sodium heparin and were centrifuged at 3300 rpm for 10 min and plasma were collected. Celecoxib plasma concentrations were determined with a validated HPLC assay. Pharmacokinetic (PK) parameters were determined by a Microsoft™ Excel add-in software. Results are shown in Table 12 and were compared with the published PK parameters for a Celebrex® 200 mg capsule.

TABLE 12
Comparison of PK of celecoxib after administration of two
different liquid suspensions and a capsule in a human volunteer.

| | Human Volunteer (Current Study) | | | |
|---|---|---|---|---|
| | | Liquid | Liquid | Celebrex® Capsule |
| Celecoxib | Reference | Suspension | Suspension | 200 mg |
| Pk | Capsule | # C-18 | # C-19 | Published |
| Parameter | 200 mg celecoxib | 200 mg celecoxib | 200 mg celecoxib | Values^{a} |
| Tmax, h | 2 | 1 | 1 | 2.9 ± 1.4 |
| t½, h | 5 | 8 | 8 | 12.9 ± 7.7 |
| Cmax, ng/ml | 579 | 860 | 719 | 704 ± 330 |
| AUC 0-t, | 5996 | 4920 | 5600 | 7267 ± 2078 |
| ng/ml*h | | | | |

^{a}Chang WK et al., (2015) Int J Bioanal Methods Bioequival Stud, 2(2), 34-40.

One of the advantages for a liquid oral formulation of celecoxib is the rapid onset of pain relief on first use. The prerequisite for this is that celecoxib reaches Cₘₐₓ (maximum plasma concentration) in a shortest period of time i.e. tₘₐₓ (time to reach Cₘₐₓ) for a suspension is appreciably less than that for a capsule. Results in Table 12 suggest that in the formulation according to the above examples this could be achieved. In a direct comparison between a suspension C-18 or celecoxib suspension C-19 according to the invention and a capsule containing the same dose, the time for maximum plasma concentration on a single dose of 200 mg celecoxib for both the suspensions is tₘₐₓ=1 h for both the suspensions as against tₘₐₓ=2 h-2.9 h for the capsule.

Regulatory agencies, like the Food and Drug Administration and the European Medicines Evaluation Agency, consider a product bioequivalent if the ratio of Cₘₐₓ, and AUC₀₋ₜ (area under plasma concentration time curve from time zero to time of last quantifiable concentration) values for the test and reference products were within the acceptance range of 80% to 125%. Results in Table 12 suggest that in the formulations, according to the above examples, the bioequivalence could be achieved. In a direct comparison between a suspension C-19, according to the invention, and a capsule containing the same dose in a human volunteer, the values of Cₘₐₓ, and AUC₀₋ₜ were within this range and comparable to published values.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the Applicant.

What is claimed is:

1. An aqueous pharmaceutical liquid suspension for oral administration to a mammal in need thereof comprising:
a) a pharmaceutically acceptable salt selected from the group consisting of celecoxib and allopurinol;
b) crospovidone;
c) glycerol; and
d) at least one of each of the group consisting of an aqueous buffer system, a suspending agent, a wetting agent, a sweetening agent, a preservative, and a pharmaceutically acceptable liquid carrier.

2. The liquid pharmaceutical composition of claim 1, wherein the celecoxib, pharmaceutically acceptable salt thereof is present in the liquid at about 0.5 to about 3.0 percent w/v.

3. The liquid pharmaceutical composition of claim 2, wherein the celecoxib, pharmaceutically acceptable salt thereof is present in the liquid at about 0.75 to about 2.0 percent w/v.

4. The liquid pharmaceutical composition of claim 3, wherein the celecoxib, pharmaceutically acceptable salt thereof is present in the liquid at about 1.0 percent w/v.

5. The liquid pharmaceutical composition of claim 1, wherein the allopurinol, pharmaceutically acceptable salt thereof is present in the liquid at about 1.0 to about 3.0 percent w/v.

6. The liquid pharmaceutical composition of claim 5, wherein the allopurinol, pharmaceutically acceptable salt thereof is present in the liquid at about 1.5 to about 2.5 percent w/v.

7. The liquid pharmaceutical composition of claim 1, wherein the particle size distribution of the pharmaceutically acceptable salt of the celecoxib and allopurinol particles in suspension is greater than about 3 micron to less than about 50 micron.

8. The liquid pharmaceutical composition of claim 7, wherein the particle size distribution of the pharmaceutically acceptable salt of the celecoxib and allopurinol particles in suspension includes at least 90% of the particles that are smaller than about 50 micron,

9. The liquid pharmaceutical composition of claim 1, wherein the crospovidone is present in the liquid at about 1 to about 30 percent w/v.

10. The liquid pharmaceutical composition of claim 9, wherein the crospovidone is present in the liquid at about 1 to about 10 percent w/v.

11. The liquid pharmaceutical composition of claim 10, wherein the crospovidone is present in the liquid at about 5 percent w/v.

12. The liquid pharmaceutical composition of claim 1, wherein the glycerol is present in the liquid at about 0.5 to about 50 percent w/v.

13. The liquid pharmaceutical composition of claim 12, wherein the glycerol is present in the liquid at about 1 to about 10 percent w/v.

14. The liquid pharmaceutical composition of claim 1, wherein a suspending agent is selected from a group consisting of xanthan gum, guar gum, magnesium stearate and microcrystalline cellulose.

15. The liquid pharmaceutical composition of claim 1, wherein one or more wetting agents are selected from a group consisting of polyethylene glycols, sorbitan monolaurate, polysorbate 80, polysorbate 20, and sodium lauryl sulfate.

16. The liquid pharmaceutical composition of claim 1, wherein one or more sweeteners are selected from any natural or artificial sweeteners including glucose, fructose, invert sugar, sorbitol, sucrose, maltose, xylose, ribose, mannose, corn syrup solids, xylitol, mannitol, maltodextrins, saccharin, aspartame, and sucralose.

17. The liquid pharmaceutical composition according to claim 1, wherein one or more preservatives are selected from benzoic acid, sodium benzoate, potassium sorbate, cresol, cetrimide, citric acid and sodium citrate, and alkyl hydroxybenzoates including such as methyl hydroxybenzoate, ethyl hydroxybenzoate, propyl hydroxybenzoate (as base or sodium salt).

18. The liquid pharmaceutical composition of claim 1, wherein a buffer system compromises an aqueous mixture of an acid wherein the acid is phosphoric, succinic, tartaric, lactic, or citric acid, and a base, wherein the base is trisodium citrate dehydrate, sodium hydroxide, or disodium hydrogen phosphate, for maintaining the pH in the range from 4 to 6.

19. The liquid pharmaceutical composition of claim 1, wherein the pharmaceutically acceptable liquid carrier comprises water.

20. A method of treating a mammal in need of the treatment of a disease indication treated with a composition selected from the group consisting of celecoxib and allopurinol comprising administering to the mammal an effective amount of a liquid pharmaceutical composition comprising:
a) a pharmaceutically acceptable salt selected from the group consisting of celecoxib and allopurinol;
b) crospovidone;
c) glycerol; and
d) at least one of each of the group consisting of an aqueous buffer system, a suspending agent, a wetting agent, a sweetening agent, a preservative, and a pharmaceutically acceptable liquid carrier.
